(19) United States
(12) Patent Application Publication
(10) Pub. No.: US 2008/0141467 A1
(43) Pub. Date: Jun. 19, 2008

(54) Colouring of Hair

(76) Inventor: Arthur Paul Wood, Queensland (AU)

Correspondence Address: HOFFMAN WASSON & GITLER, P.C;CRYSTAL CENTER 2, SUITE 522, 2461 SOUTH CLARK STREET, ARLINGTON, VA, 22202-3843, US

(21) Appl. No.: 11/883,834

(22) PCT Filed: Feb. 8, 2006
(86) PCT No.: PCT/AU06/00158

§ 371 Date: Feb. 26, 2008

(30) Foreign Application Priority Data
Feb. 8, 2005 (AU) 2005900554

(51) Int. Cl. A61K 8/00; B05B 5/025; A45D 7/00

(52) U.S. Cl. 8/405; 118/621; 132/200; 132/212

(57) ABSTRACT

An apparatus for colouring hair comprises a vacuum chamber for housing hair, a vacuum pump for creating a vacuum in the vacuum chamber, a charger for electrically charging hair which is contained in the vacuum chamber, a metal electrode, and a vaporiser for vaporising the electrode in the vacuum chamber. Hair is able to be coated with metal from the electrode by inserting the hair into the vacuum chamber, creating a vacuum in the chamber with the vacuum pump, charging the hair with the charger, and by vaporising the electrode with the vaporiser.

### FIELD OF THE INVENTION

The present invention relates generally to hair colouring and, in particular, to improvements relating to the colouring of hair.

Although the invention will be described with particular reference to the colouring of hair which grows on a human head, it will be appreciated that this is by way of example only and that the invention may be used in relation to the colouring other hair.

### BRIEF DESCRIPTION OF THE PRIOR ART

The colouring of hair by dying or other processes is a popular method of altering one's appearance. Modern hair dying or colouring methods typically involve applying a concentrated liquid chemical dying or colouring agent such as hydrogen peroxide to one's hair. Such agents chemically react with hair in such a way that the pigment or colour of the hair is altered.

Although modern hair dying or colouring methods generally achieve satisfactory results, it would be desirable to have a method of altering hair colour which does not alter hair pigment and which enhances hair lustre, decreases the amount of hair care required, and increases personal hygiene.

### SUMMARY OF THE INVENTION

It is an object of the present invention to overcome, or at least ameliorate, one or more of the deficiencies of the prior art mentioned above, or to provide the consumer with a useful or commercial choice.

Other objects and advantages of the present invention will become apparent from the following description, taken in connection with the accompanying illustration, wherein, by way of illustration and example, a preferred embodiment of the present invention is disclosed.

According to a first broad aspect of the present invention there is provided an apparatus for colouring hair, the apparatus comprising a vacuum chamber for housing hair, a vacuum pump for creating a vacuum in the vacuum chamber, a charger for electrically charging hair which is contained in the vacuum chamber, a metal electrode, and a vaporiser for vaporising the electrode in the vacuum chamber, wherein hair is able to be coated with metal from the electrode by inserting the hair into the vacuum chamber, creating a vacuum in the chamber with the vacuum pump, charging the hair with the charger, and by vaporising the electrode with the vaporiser.

Placing electrically charged hair in a vacuum and then vaporising a metal electrode in the vacuum results in the metal being deposited on the surface of the charged hair so that the hair is coated with the metal. If hair is coated in the aforementioned manner with an appropriate metal such as gold, silver, platinum or palladium for example, the lustre of the hair can be enhanced and the colour of the hair can be altered without altering the pigmentation of the hair.

For a person to maintain the lustre of their hair, they usually must wash and brush their hair on a regular basis. This can require a significant amount of time and effort, particularly if the hair is long and thick. By coating their hair with metal in the aforementioned manner, a person can reduce the amount of time and effort that they need to devote to caring for and maintaining the appearance of their hair.

It is preferred that the apparatus is adapted to coat hair on a person's head with metal from the electrode.

The vacuum chamber is preferably adapted to be mounted on a person's head such that hair which extends therefrom is housed inside the chamber.

In a particular preferred form, the vacuum chamber includes an opening for receiving the scalp portion of a person's head such that the scalp portion and the hair which extends therefrom are located inside the chamber. Preferably, a hermetic seal is formed between the chamber and the person's head around the scalp portion thereof such that the seal extends along the forehead of the person's head and the back of the person's neck.

The vacuum pump may be provided by any suitable pump which is able to create a vacuum inside the vacuum chamber. Preferably, the vacuum pump is an electrically-powered vacuum pump.

The charger may be provided by any suitable charger which is able to charge the hair which is housed in the vacuum chamber with an electric charge.

The hair may be more uniformly coated with metal from the electrode if the individual hair strands are separated from each other. In a particular preferred form, the charger is able to charge the hair which is contained in the vacuum chamber with an electric charge of sufficient magnitude to cause individual strands of the hair to separate from each other. The degree to which the individual hair strands are separated from each other may be enhanced if the magnitude of the charge is sufficient to cause the individual strands of hair to be substantially upstanding from the surface of the scalp or other surface from which they project.

The metal electrode may be of any suitable size and shape. Preferably, there is sufficient metal in the electrode to coat all of the hair in the vacuum chamber.

The electrode may be fabricated from any suitable metal. For example, the metal may be a base metal, a precious metal or a metal alloy. If the metal is a precious metal or a precious metal alloy, the metal or metal alloy is preferably selected from the group of metals which includes gold, silver, platinum, and palladium.

In a particular preferred embodiment of the apparatus, the vaporiser is of a type which is able to vaporise the metal electrode in the vacuum chamber by passing an electric current through the electrode.

According to a second broad aspect of the present invention there is provided a method of colouring hair, the method comprising the steps of:

(i) inserting hair into a vacuum chamber;

(ii) creating a vacuum in the vacuum chamber;

(iii) electrically charging the hair; and

(iv) vaporising a metal electrode in the vacuum chamber.

The method preferably also includes the step of washing the hair prior to colouring the hair.

Either during or after the washing process, the hair may be bathed in an acid or alkaline-based solution to improve adhesion between the metal and the hair and to minimise the amount of metal which is required to coat the hair.

It is preferred that the hair is dried prior to being coloured using the above-mentioned method. In a particular preferred form, the hair is dried in purified heated air.

Preferably, after being washed and dried, and before being coloured, the hair is immersed in a sulphate solution which includes compounds or elements such as iodine or copper so as to increase the electromagnetic attraction between the hair and the vaporised metal from the metal electrode.

The metal electrode may be vaporised by passing an electric current of a sufficient magnitude through it. Alternatively, the metal electrode may be vaporised by thermal means including for example, the application of thermal energy to the electrode by convection conduction or by radiation.

### BRIEF DESCRIPTION OF THE ILLUSTRATION

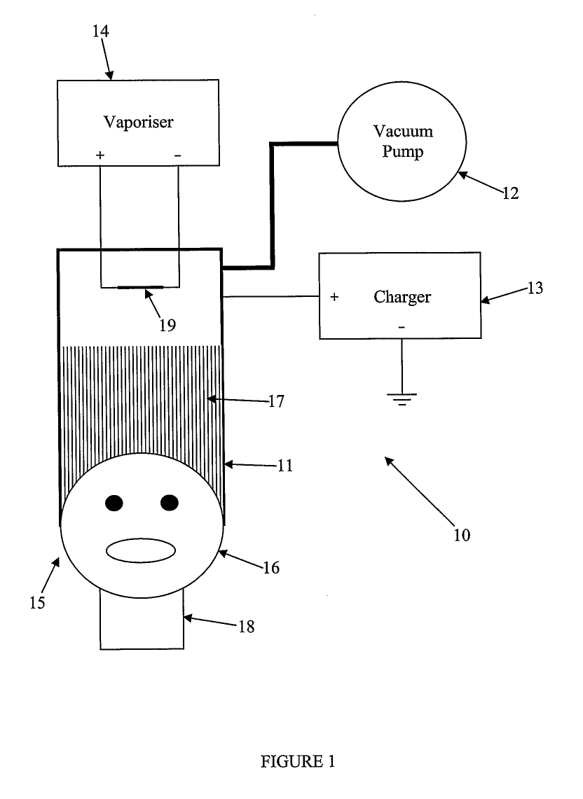

In order that the invention may be more fully understood and put into practice, a preferred embodiment thereof will now be described with reference to FIG. 1 of the accompanying illustration which depicts an apparatus for colouring hair according to the preferred embodiment of the present invention.

### DETAILED DESCRIPTION OF THE PREFERRED EMBODIMENT

An apparatus 10 for colouring hair in accordance with the preferred embodiment of the present invention is depicted in FIG. 1 of the illustration.

Apparatus 10 includes a vacuum chamber 11, a vacuum pump 12, a charger 13, and a vaporiser 14.

Vacuum chamber 11 includes an opening 15 for receiving the scalp portion of a person's head 15 such that the scalp portion and the hair 17 extending therefrom are located inside the chamber 11, and such that a hermetic seal is formed between the chamber 11 and the person's head 16 around the scalp portion thereof such that the seal extends along the forehead of the person's head 15 and the back of the person's neck 18. Hair 17 should be dry before the vacuum chamber 11 is placed on the person's head 16.

After the chamber 11 has been placed on the person's head 16 in the aforementioned manner, vacuum pump 12 is operated to evacuate air from the interior of the chamber 11 to create a vacuum therein.

Charger 13 is then operated to charge the hair 17 which extends from the person's head 16 and which is located inside the chamber 11 with an electric charge of sufficient magnitude so that a respective electrical field surrounds each strand of hair 17. Each strand of hair 17 is charged with an electric charge of the same polarity so that the electrical fields which surround the strands of hair 17 causes the hair strands to repel each other and to be upstanding from the scalp of the person's head 16 as depicted in FIG. 1.

After hair 17 has been charged, vaporiser 14 is operated to vaporise a metal electrode in the form of a wire filament 19 which is made from a base metal or precious metal such as gold, silver, platinum, or palladium, or metal alloy having a colour with which it is desired to re-colour the hair 17 with. Vaporiser 14 vaporizes the wire filament 19 by passing an electric current of sufficient magnitude through the filament 19. The metal is then uniformly deposited on the surface of each individual strand of hair 17 inside the vacuum chamber 11 such that each hair strand is coated with the metal. The colour of the coated hair strands corresponds with the colour of the metal.

The vacuum chamber 11 is removed from the person's head 16 once the hair 17 has been coated with the vaporised metal.

Hair 17 would usually be washed prior to being coated with metal. Also, either after or during the washing process, hair 17 may be bathed in an acid or alkaline-based solution to improve adhesion between the metal and the hair 17 so that the amount of metal which is required to coat the hair 17 is minimised. Hair 17 would then be dried in purified heated air before being housed in the vacuum chamber 11.

The method of colouring hair as just described enhances hair lustre, decreases the amount of hair care required, and increases personal hygiene.

Various colour tones can be achieved for example by varying the metal or metal alloy which the filament 19 is fabricated from, or by altering the colour of the aforementioned metal or metal alloy by a dying process.

The colour of the hair 17 after being treated using the method as just described would be substantially permanent. If necessary, the hair 17 may be re-treated any number of times using the same method.

The metal or metal alloy which coats the hair 17 may be removed from the hair 17 by using a reverse anode collection process.

Throughout the specification and the claims, unless the context requires otherwise, the term “comprise”, or variations such as “comprises” or “comprising”, will be understood to apply the inclusion of the stated integer or group of integers but not the exclusion of any other integer or group of integers.

Throughout the specification and claims, unless the context requires otherwise, the term “substantially” or “about” will be understood to not be limited to the value for the range qualified by the terms.

It will be appreciated by those skilled in the art that variations and modifications to the invention described herein will be apparent without departing from the spirit and scope thereof. The variations and modifications as would be apparent to persons skilled in the art are deemed to fall within the broad scope and ambit of the invention as herein set forth.

It will be clearly understood that, if a prior art publication is referred to herein, that reference does not constitute an admission that the publication forms part of the common general knowledge in the art in Australia or in any other country.

1. An apparatus for colouring hair, the apparatus comprising a vacuum chamber for housing hair, a vacuum pump for creating a vacuum in the vacuum chamber, a charger for electrically charging hair which is contained in the vacuum chamber, a metal electrode, and a vaporiser for vaporising the electrode in the vacuum chamber, wherein hair is able to be coated with metal from the electrode by inserting the hair into the vacuum chamber, creating a vacuum in the chamber with the vacuum pump, charging the hair with the charger, and by vaporising the electrode with the vaporiser.

2. The apparatus of claim 1, wherein the apparatus is adapted to coat hair on a person's head with metal from the electrode.

3. The apparatus of claim 1, wherein the vacuum chamber is adapted to be mounted on a person's head such that hair which extends therefrom is housed inside the vacuum chamber.

4. The apparatus of claim 3, wherein the vacuum chamber includes an opening for receiving the scalp portion of the person's head such that the scalp portion and the hair which extends therefrom are located inside the chamber.

5. The apparatus of claim 4, wherein a hermetic seal is formed between the vacuum chamber and the person's head around the scalp portion thereof such that the seal extends along the forehead of the person's head and the back of the person's neck.

6. The apparatus of claim 1, wherein the vacuum pump is an electrically-powered vacuum pump.

7. The apparatus of claim 1, wherein the charger is able to charge the hair which is contained in the vacuum chamber with an electric charge of sufficient magnitude to cause individual strands of the hair to separate from each other.

8. The apparatus of claim 7, wherein the charger is able to charge the hair which is contained in the vacuum chamber with an electric charge of sufficient magnitude to cause individual strands of the hair to substantially standup from the surface of the scalp or other surface from which they project.

9. The apparatus of claim 1, wherein there is sufficient metal in the electrode to coat all of the hair in the vacuum chamber.

10. The apparatus of claim 1, wherein the electrode is preferably fabricated from a metal or a metal alloy which is selected from the group of metals which includes gold, silver, platinum and palladium.

11. The apparatus of claim 1, wherein the vaporiser vaporizes the metal electrode by passing an electric current through the electrode.

12. A method of colouring hair, the method comprising the steps of:
(i) inserting hair into a vacuum chamber;
(ii) creating a vacuum in the vacuum chamber;
(iii) electrically charging the hair; and
(iv) vaporising a metal electrode in the vacuum chamber.

13. The method of claim 12, wherein the method also includes the step of washing the hair.

14. The method of claim 12, wherein the method also includes the step of bathing the hair in an acid or alkaline-based solution.

15. The method of claim 13, wherein the method also includes the step of drying the hair.

16. The method of claim 15, wherein the hair is dried in purified heated air.

17. The method of claim 15, wherein the method also includes the step of immersing the hair in a sulphate solution.

18. The method of claim 12, wherein the step of vaporising the metal electrode involves passing an electric current through the electrode.

19. The method of claim 12, wherein the step of vaporising the metal electrode involves applying thermal energy to the electrode.
